(19) United States
(12) Patent Application Publication
(10) Pub. No.: US 2007/0015761 A1
(43) Pub. Date: Jan. 18, 2007

(54) Tetrahydronaphthalene derivatives, process for their production and their use as anti-inflammatory agents

(76) Inventors: Anne Mengel, Berlin (DE); Konrad Krolikiewicz, Berlin (DE); Stefan Baeurle, Berlin (DE); Heike Schaecke, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/399,610

(22) Filed: Apr. 7, 2006

Related U.S. Application Data
(60) Provisional application No. 60/671,108, filed on Apr. 14, 2005.

(30) Foreign Application Priority Data
Apr. 14, 2005 (DE) 10 2005 017 316.0

(51) Int. Cl. A61K 31/517; A61K 31/502; A61K 31/498; A61K 31/4706; A61K 31/4745; A61K 31/4184; A61K 31/416; A61K 31/405

(52) U.S. Cl. 514/247; 544/235; 544/294; 544/353; 544/105; 546/122; 546/117; 546/153; 546/147; 546/141; 548/471; 548/307.4; 514/248; 514/266.4; 514/249; 514/309; 514/312; 514/300; 514/303; 514/406; 514/394; 514/419

(57) ABSTRACT

The invention relates to polysubstituted tetrahydronaphthalene derivatives of formula (I), 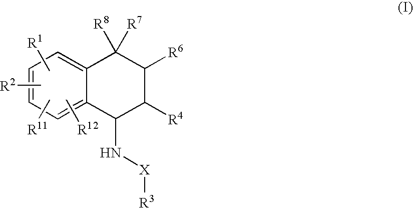 process for their production and their use as anti-inflammatory agents.

This application claims the benefit of the filing date of U.S. Provisional Application Ser. No. 60/671,108 filed Apr. 14, 2005, which is incorporated by reference herein.

### INTRODUCTION

The invention relates to tetrahydronaphthalene derivatives, process for their production and their use as anti-inflammatory agents.

Open-chain, non-steroidal anti-inflammatory agents are known from the prior art (DE 100 38 639 and WO 02/10143). In the experiment, these compounds show dissociations of action between anti-inflammatory and undesirable metabolic actions and are superior to the previously described nonsteroidal glucocorticoids or exhibit at least just as good an action.

In this invention, other nonsteroidal anti-inflammatory agents are made available.

### BRIEF DESCRIPTION OF THE INVENTION

This invention relates to compounds of general formula (I), 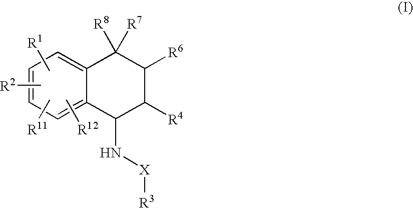
in which
R¹ and R², independently of one another, are a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group,
or R¹ and R² together form a group that is selected from the groups —O—(CH₂)ₙ-O—, —O—(CH₂)ₙ-CH₂—, —O—CH═CH—, —(CH₂)ₙ₊₂-, —NH—(CH₂)ₙ₊₁-, —N(C₁-C₃-alkyl)-(CH₂)ₙ₊₁-, and —NH—N═CH—, whereby n=1 or 2, and the terminal oxygen atoms and/or carbon atoms and/or nitrogen atoms are linked to directly adjacent ring-carbon atoms,
R¹¹ is a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a (C₁-C₁₀)-alkyl group that optionally is substituted by 1 to 3 hydroxy groups, 1 to 3 halogen atoms, and/or 1 to 3 (C₁-C₅)-alkoxy groups, an optionally substituted (C₃-C₇)-cycloalkyl group, an optionally substituted heterocyclyl group, an optionally substituted aryl group, a monocyclic or bicyclic heteroaryl group that optionally is substituted by one or more groups, which independently of one another are selected from
   (C₁-C₅)-alkyl groups, which themselves optionally can be substituted by 1 to 3 hydroxy groups or 1 to 3 —COOR¹³ groups,
   (C₁-C₅)-alkoxy groups,
   halogen atoms, hydroxy groups, —NR⁹R^{9a} groups, and
   exomethylene groups,
   and that contains 1 to 4 nitrogen atoms and/or 1 to 2 oxygen atoms and/or 1 to 2 sulfur atoms and/or 1 to 2 keto groups, whereby this group is linked via any position to group X and optionally can be hydrogenated at one or more sites,
R⁴ is a hydroxy group, an —OR¹⁰ group or an —O(CO)R¹⁰ group,
R⁶ is a hydrogen atom, a halogen atom, or an optionally substituted (C₁-C₁₀)-alkyl group,
R⁷ and R⁸, independently of one another, mean a hydrogen atom, a halogen atom, an optionally substituted (C₁-C₁₀)-alkyl group, a cyano group, together a (C₁-C₁₀)-alkylidene group or together with the carbon atom of the tetrahydronaphthalene system an optionally substituted (C₃-C₆)-cycloalkyl ring; or
R⁶ and R⁷ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C_{1-C}₅)-alkoxy groups, and/or 1 to 4 halogen atoms; or
R¹ and R⁸ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms;
R⁹ and R^{9a}, independently of one another, are a hydrogen atom, a (C₁-C₅)-alkyl group or —(CO)—(C₁-C₅)-alkyl,
R¹⁰ means a (C₁-C₁₀)-alkyl group or any hydroxy protective group,
R¹³ means a hydrogen atom or a (C₁-C₅)-alkyl group, and
X means a bond or a group —C(═O)—, —C(═S)—, —C(═O)—NH—, —C(═S)—NH—, —S(O)ₘ- (whereby m=1 or 2), —C(═O)—O—, —C(═S)—O— or a group —(CH₂)ₚ- (whereby p=1, 2 or 3), whereby, if X contains a carbonyl or thiocarbonyl function, this function is bonded to the group —NH— in general formula (I),
   in the form of any stereoisomer or a mixture of stereoisomers; or as a pharmacologically harmless salt or derivative.

In addition, this invention relates to a process for the production of compounds of general formula (I), as described herein.

In addition, this invention relates to pharmaceutical compositions that comprise one or more compounds of general formula (I) in combination with one or more pharmaceutical vehicles or adjuvants.

This invention relates, moreover, to the use of compounds of general formula (I) for the production of pharmaceutical compositions with an anti-inflammatory action.

This invention relates, moreover, to compounds of general formula (II) 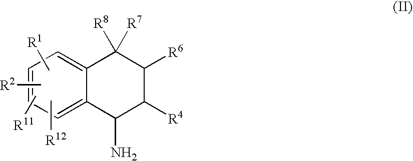
in which substituents R¹ to R¹² have the above-indicated meanings, as well as the use of these compounds for the production of compounds of general formula (I).

This invention relates, moreover, to compounds of general formula (IV) 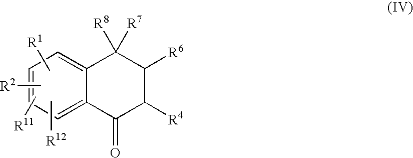
in which substituents R¹ to R¹² have the above-indicated meanings, as well as the use of these compounds for the production of compounds of general formula (I).

### In-Depth Description of the Invention

Definitions

The designation halogen atom or halogen means a fluorine, chlorine, bromine, or iodine atom. Preferred is a fluorine, chlorine or bromine atom.

The alkyl groups that are mentioned in the definitions of general formula (I) can be straight-chain or branched and stand for, for example, a methyl, ethyl, n-propyl, iso-propyl, n-butyl, iso-butyl, tert-butyl or n-pentyl, 2,2-dimethylpropyl, 2-methylbutyl or 3-methylbutyl group, as well as the hexyl, heptyl, nonyl, or decyl group and their arbitrarily branched derivatives. Alkyl groups that contain 1 to 10, 1 to 8, or 1 to 5 carbon atoms are preferred. A methyl or ethyl group is especially preferred.

The above-mentioned alkyl groups can optionally be substituted by 1 to 5, preferably 1 to 3, groups, which, independently of one another, are selected from hydroxy, cyano, nitro, —COOR¹³, C₁-C₅-alkoxy groups, halogen, —NR⁹R^{9a}, and a partially or completely fluorinated C₁-C₃-alkyl group. The alkyl groups can preferably be substituted by 1 to 3 halogen atoms and/or 1 to 3 hydroxy groups and/or 1 to 3 cyano groups and/or 1 to 3 —COOR¹³ groups. Fluorine atoms, hydroxy, methoxy and/or cyano groups represent an especially preferred subgroup of substituents.

1 to 3 hydroxy groups and/or 1 to 3 —COOR¹³ groups are another preferred group of substituents for the alkyl groups. Especially preferred in this case are the hydroxy groups.

For a partially or completely fluorinated alkyl group, for example, the following partially or completely fluorinated groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, and pentafluoroethyl. Of the latter, the trifluoromethyl group or the pentafluoroethyl group is preferred. The completely fluorinated group is also named perfluoroalkyl group. The reagents, which optionally can be used during the synthesis, are commercially available, or the published syntheses of the corresponding reagents belong to the prior art, or published syntheses can be used analogously.

The alkenyl groups have at least one C═C-double bond and can be straight-chain or branched. Alkenyl groups with 2 to 8 carbon atoms are preferred.

The alkinyl groups have at least a C≡C-triple bond and can be straight-chain or branched. Alkinyl groups with 2 to 8 carbon atoms are preferred.

The alkoxy groups that are mentioned in the definitions of general formula (I) can be straight-chain or branched and stand for, for example, a methoxy, ethoxy, n-propoxy, iso-propoxy, n-butoxy, iso-butoxy, tert-butoxy or n-pentoxy, 2,2-dimethylpropoxy, 2-methylbutoxy or 3-methylbutoxy group. C₁-C₅- as well as C₁-C₃-, C₁-C₈-, and C₁-C₁₀-alkoxy groups are preferred. A methoxy or ethoxy group is especially preferred.

The alkylthio groups that are mentioned in the definitions of general formula (I) can be straight-chain or branched and stand for, for example, a methylthio, ethylthio, n-propylthio, iso-propylthio, n-butylthio, iso-butylthio, tert.-butylthio or n-pentylthio, 2,2-dimethylpropylthio, 2-methylbutylthio or 3-methylbutylthio group. C₁-C₅-Alkylthio groups are preferred. A methylthio or ethylthio group is especially preferred.

On their alkyl groups, the above-described alkoxy and alkylthio groups can carry the same substituents that were already further described above for the alkyl groups in general. Preferred substituents for alkoxy and alkylthio groups are selected, independently of one another, from halogen (in particular fluorine and/or chlorine), hydroxy and cyano.

Substituent —NR⁹R^{9a} means, for example, —NH₂, —NH(CH₃), —N(CH₃)₂, —NH(C₂H₅), —N(C₂H₅)₂, —NH(C₃H₇), —N(C₃H₇)₂, —NH(C₄H₉), —N(C₄H₉)₂, —NH(C₅H₁₁), —N(C₂H₅), —NH(CO)CH₃, —NH(CO)C₂H₅, —NH(CO)C₃H₇, —NH(CO)C₄H₉, or —NH(CO)C₅H₁₁.

The (C₃-C₇)-cycloalkyl group means a saturated cyclic group with 3 to 7 ring-carbon atoms—such as, for example, cyclopropyl, methylcyclopropyl, cyclobutyl, methylcyclobutyl, cyclopentyl, methylcyclopentyl, cyclohexyl, methylcyclohexyl, cycloheptyl, or methylcycloheptyl—that optionally is substituted by one or more groups—selected from hydroxy groups, halogen atoms, (C₁-C₅)-alkyl groups, (C₁-C₅)-alkoxy groups, —NR⁹R^{9a} groups, —COOR¹³ groups, —CHO, and cyano.

An alkylidene or exoalkylidene group is defined as a group with 1 to 10 carbon atoms that is bonded to the system (ring or chain) via an exo-double bond. (C₁-C₅)- and (C₁-C₃)-alkylidene is preferred; exomethylene is especially preferred.

The heterocyclyl group is a cyclic, non-aromatic group that contains one or more heteroatoms and can be, for example, pyrrolidine, imidazolidine, pyrazolidine, or piperidine. Perhydroquinoline and perhydroisoquinoline are also part of the heterocyclyl groups according to the invention.

As substituents for heterocyclyl and heteroaryl groups, for example, substituents from the following group are considered: optionally substituted C₁-C₅-alkyl group, hydroxy, C₁-C₅-alkoxy, —NR⁹R^{9a}, halogen, cyano, —COOR¹³, and —CHO. The substituents can optionally also be bonded to the nitrogen atom of the heterocyclyl or heteroaryl group; N-oxides are included in the definition.

In terms of the invention, aryl groups are aromatic or partially aromatic carbocyclic groups with 6 to 14 carbon atoms, which have a ring, such as, e.g., phenyl or phenylene, or several condensed rings, such as, e.g., naphthyl or anthranyl. By way of example, phenyl, naphthyl, tetralinyl, anthranyl, indanyl and indenyl can be mentioned. The optionally substituted phenyl group and the naphthyl group are preferred.

The aryl groups can be substituted at any suitable site that results in a stable compound by one or more radicals from the group of hydroxy, halogen, C₁-C₅-alkyl that optionally is substituted by 1 to 3 hydroxy groups or —COOR¹³ groups, C₁-C₅-alkoxy, cyano, —CF₃, and nitro.

The aryl groups can be partially hydrogenated and can also carry keto and/or exoalkylidene groups in addition to or as an alternative to the above-cited substituents. Partially hydrogenated phenyl is defined as, e.g., cyclohexadienyl, cyclohexenyl, or cyclohexyl. A partially hydrogenated substituted naphthalene system is, for example, 1-tetralone or 2-tetralone.

A (C₁-C₈)alkylaryl group is an aryl group, as it is already described above, which is linked to the ring system via a straight-chain or branched (C₁-C₈)-alkyl unit (as defined above).

A (C₂-C₈)alkenylaryl group is an aryl group, as it is already described above, which is linked to the ring system via a straight-chain or branched (C₂-C₈)-alkenyl unit (as defined above).

A (C₂-C₈)alkinylaryl group is an aryl group, as it is already described above, which is linked to the ring system via a straight-chain or branched (C₂-C₈)-alkinyl unit (as defined above).

The monocyclic or bicyclic heteroaryl group can optionally contain 1 to 9 groups, selected from nitrogen atoms, oxygen atoms, sulfur atoms or keto groups, of which a maximum of 4 nitrogen atoms, a maximum of 2 oxygen atoms, a maximum of 2 sulfur atoms and/or a maximum of 2 keto groups can be contained. Any subcombination of these groups is possible. The heteroaryl group can be hydrogenated at one or more sites.

Monocyclic heteroaryl groups can be, for example, pyridine, pyrazine, pyrimidine, pyridazine, triazine, azaindolizine, 2H- and 4H-pyran, 2H- and 4H-thiopyran, furan, thiophene, 1H- and 4H-pyrazole, 1H- and 2H-pyrrole, oxazole, thiazole, furazan, 1H- and 4H-imidazole, isoxazole, isothiazole, oxadiazole, triazole, tetrazole, or thiadiazole.

Bicyclic heteroaryl groups can be, for example, a phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzo[b]thienyl, benzo[c]thienyl, benzimidazolyl, dihydroisoquinolinyl, dihydroquinolinyl, benzoxazinonyl, phthalazinonyl, dihydrophthalazinonyl, quinolinyl, isoquinolinyl, quinolonyl, isoquinolonyl, quinazolinyl, quinoxalinyl, cinnolinyl, phthalazinyl, dihydrophthalazinyl, 1,7- or 1,8-naphthyridinyl, coumarinyl, isocoumarinyl, indolizinyl, isobenzofuranyl, azaindolyl, azaisoindolyl, furanopyridyl, furanopyrimidinyl, furanopyrazinyl, pyrazolo[1,5-a]pyridinyl, furanopyridazinyl, dihydrobenzofuranyl, dihydrofuranopyridyl, dihydrofuranopyrimidinyl, dihydrofuranopyrazinyl, dihydrofuranopyridazinyl, or dihydrobenzofuranyl group.

If the heteroaryl groups are partially or completely hydrogenated, compounds of general formula (I), in which R³ means tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylidenaminyl, chromanyl, isochromanyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, or 2,2-dihydro-1H-pyrrolo[2,3-b]pyridin-3-onyl, are part of this invention.

The monocyclic or bicyclic heteroaryl group optionally can be substituted by one or more substituents, selected from C₁-C₅-alkyl groups, C₁-C₅-alkoxy groups, halogen atoms, and/or exomethylene groups that optionally are substituted by 1 to 3 hydroxy groups or 1 to 3 —COOR¹³ groups. The substituents can, if possible, optionally also be directly bonded to the heteroatom (e.g., to the nitrogen atom). N-Oxides are also part of this invention.

A (C₁-C₈)alkylheteroaryl group is a heteroaryl group, as it is already described above, which is linked to the ring system via a straight-chain or branched (C₁-C₈)-alkyl unit (as defined above).

A (C₂-C₈)alkenylheteroaryl group is a heteroaryl group, as it is already described above, which is linked to the ring system via a straight-chain or branched (C₂-C₈)-alkenyl unit (as defined above).

A (C₂-C₈)alkinylheteroaryl group is a heteroaryl group, as it is already described above, which is linked to the ring system via a straight-chain or branched (C₂-C₈)-alkinyl unit (as defined above).

A (C₁-C₈)alkylheterocyclyl group is a heterocyclyl group, as it is already described above, which is linked to the ring system via a straight-chain or branched (C₁-C₈)-alkyl unit (as defined above).

A (C₂-C₈)alkenylheterocyclyl group is a heterocyclyl group, as it is already described above, which is linked to the ring system via a straight-chain or branched (C₂-C₈)-alkenyl unit (as defined above).

As hydroxy protective groups, all hydroxy protective groups that are commonly known to one skilled in the art, in particular silyl ether or ester of organic C₁-C₁₀ acids, C₁-C₅ ether, benzyl ether or benzyl ester, are suitable. The common hydroxy protective groups are described in detail in T. W. Greene, P. G. M. Wuts “Protective Groups in Organic Synthesis,” 2nd Edition, John Wiley & Sons, 1991). The protective groups are preferably alkyl-, aryl- or mixed alkylaryl-substituted silyl groups, e.g., the trimethylsilyl (TMS), triethylsilyl (TES), tert-butyldimethylsilyl (TBDMS), tert-butyldiphenylsilyl (TBDPS) or triisopropylsilyl groups (TIPS) or other standard hydroxy protective groups (e.g., methoxymethyl, methoxyethoxymethyl, ethoxyethyl, tetrahydrofuranyl, or tetrahydropyranyl groups).

The compounds of general formula (I) according to the invention can be present as stereoisomers because of the presence of asymmetry centers. Subjects of this invention are all possible diastereomers, both as racemates and in enantiomer-pure form. The term stereoisomers also comprises all possible diastereomers and regioisomers and tautomers (e.g., keto-enol tautomers), in which the stereoisomers according to the invention can be present, which thus are also subjects of the invention.

The compounds according to the invention can also be present in the form of salts with pharmacologically harmless anions, for example in the form of hydrochloride, sulfate, nitrate, phosphate, pivalate, maleate, fumarate, tartrate, benzoate, mesylate, citrate or succinate.

Pharmacologically harmless derivatives or prodrugs of the compounds of general formula (I) are also comprised by the invention. For example, esters, ethers or amides of the compounds of general formula (I) or other compounds that metabolize in the organism to form compounds of general formula (I) are referred to as derivatives or prodrugs. Suitable compounds are cited in, for example, Hans Bundgaard (publisher), Design of Prodrugs, Elsevier, Amsterdam 1985.

### Preferred Embodiments

Those compounds in which R⁷ and R⁸, independently of one another, mean a hydrogen atom, a halogen atom, an optionally substituted (C₁-C₁₀)-alkyl group, a cyano group, together a (C₁-C₁₀)-alkylidene group or, together with the carbon atom of the tetrahydronaphthalene system, an optionally substituted (C₃-C₆)-cycloalkylring, are a subgroup of the compounds of general formula (I) according to the invention.

Those compounds in which R⁶ and R⁷ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms, are another subgroup of the compounds of general formula (I) according to the invention.

Those compounds in which R¹ and R⁸ together form an annelated five- to eight-membered, saturated or unsaturated carbocyclic or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms, are another subgroup of the compounds of general formula (I) according to the invention.

Those compounds in which R¹ and R², independently of one another, are a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group are another subgroup of the compounds of general formula (I) according to the invention.

Those compounds in which R¹ and R² together form a group that is selected from the groups —O—(CH₂)ₙ-O—, —O—(CH₂)ₙ-CH₂—, —O—CH═CH—, —(CH₂)ₙ₊₂-, —NH—(CH₂)ₙ₊₁-, —N(C₁-C₃-alkyl)-(CH₂)ₙ₊₁-, and —NH—N═CH—, whereby n=1 or 2, and the terminal oxygen atoms and/or carbon atoms and/or nitrogen atoms are linked to directly adjacent ring-carbon atoms, are another subgroup of the compounds of general formula (I) according to the invention.

Those compounds in which X is a bond or a group —C(═O)—, —C(═O)—NH—, —SO₂— or —CH₂— are a preferred group of compounds of general formula (I).

Those compounds in which R⁴ is a hydroxy group or a group —OR¹⁰ are another preferred group of compounds of general formula (I). Especially preferred in this case are those compounds in which R⁴ is a hydroxy group.

Those compounds in which R⁶ is a hydrogen atom are another preferred group of compounds of general formula (I).

Those compounds in which R⁶ is a halogen atom or an optionally substituted (C₁-C₁₀)-alkyl group are another preferred group of compounds of general formula (I).

Those compounds in which R⁷ represents a halogen atom or an optionally substituted methyl or ethyl group are another preferred group of compounds of general formula (I).

Those compounds in which R⁷ and R⁸ in each case represent a methyl group or together with the carbon atom of the tetrahydronaphthalene system form a cyclopropyl group are another preferred group of compounds of general formula (I). Especially preferred in this case are the compounds in which R⁷ and R⁸ in each case represent a methyl group.

Those compounds in which R³ means an optionally substituted aryl or heteroaryl group are another preferred group of compounds of general formula (I). Especially preferred in this case are those compounds in which the aryl or heteroaryl group is selected from the group that consists of naphthyl, benzofuranyl, pyrazolo[1,5-a]pyridinyl, phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, chromanyl, isochromanyl, quinazolinyl, quinoxalinyl, cinnolinyl, phthalazinyl, 1,7- or 1,8-naphthyridinyl, dihydroindolonyl, dihydroisoindolonyl, benzimidazole or indolyl. Especially preferred in this case are the groups pyrimidinyl and naphthyl.

Those compounds in which substituents R¹¹ and R¹² in each case represent a hydrogen atom are another preferred group of compounds of general formula (I).

Those compounds in which R¹, R², R¹¹, and R¹², independently of one another, mean a hydrogen atom, a halogen atom, a hydroxy group, an optionally substituted (C₁-C₁₀)-alkyl group, or a (C₁-C₁₀)-alkoxy group, R³ means an optionally substituted aryl or heteroaryl group, R⁴ means a hydroxy group, an —OR¹⁰ group or an —O(CO)R¹⁰ group, R⁶ means a hydrogen atom, a halogen atom or an optionally substituted (C₁-C₁₀)-alkyl group, R⁷ and R⁸, independently of one another, mean an optionally substituted (C₁-C₁₀)-alkyl group, together a (C₁-C₁₀)-alkylidene group or together with the carbon atom of the tetrahydronaphthalene system an optionally substituted (C₃-C₆)-cycloalkyl ring, R¹⁰ means a (C₁-C₁₀)-alkyl group, and X means a bond or a group —C(═O)—, —C(═O)—NH—, —S(O)ₘ- (whereby m is equal to 1 or 2), or —(CH₂)ₚ- (whereby p is equal to 1, 2 or 3), are an especially preferred group of compounds of general formula (I), as defined above.

Those compounds in which R¹, R², R¹¹, and R¹², independently of one another, mean a hydrogen atom, a halogen atom, or a (C₁-C₁₀)-alkoxy group, R³ means an optionally substituted pyrimidinyl, naphthyl, or quinolinyl group, R⁴ means a hydroxy group, R⁶ means a hydrogen atom, R⁷ and R⁸, independently of one another, mean an optionally substituted (C₁-C₁₀)-alkyl group, in particular methyl, and X means a bond, are a quite especially preferred group of compounds of general formula (I) as defined above.

Any other possible combination of the above-mentioned subgroups and the substituents that are indicated to be preferred with their general and/or special meanings can also be considered as included by this invention.

Production Process

The compounds of general formula (I) according to the invention are accessible in various ways. The production processes that are described below also form a part of this invention.

If not otherwise indicated, the substituents that are used in the process descriptions mentioned below have the same meaning as above in the section “Brief Description of the Invention,” including the definitions that are indicated in the section “In-Depth Description of the Invention.”

The compounds according to the invention are produced first by amino compounds of general formula (II) being generated, 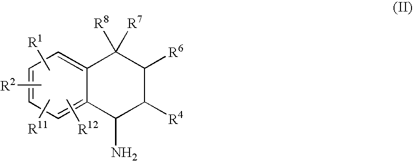
which can be reacted by various additional reactions to form compounds of general formula (I). The synthesis of the amines of general formula (II) is further described in detail below.

A process according to the invention (process A) is characterized in that a compound of general formula (II) is reacted to form a compound of general formula (I) by reaction with a compound that is selected from compounds of general formulas R³—X-Nu (whereby Nu represents a nucleofuge group), R³—N═C═O or R³—N═C═S, and optionally subsequent exchange of a carbonyl-oxygen atom for sulfur by processes that are known in the prior art.

The optional exchange of a carbonyl-oxygen atom for sulfur, mentioned in the above-described process, is known in the prior art and can be achieved, for example, by reaction with Lawesson's reagent or phosphorus pentasulfide.

For example, halogen atoms or leaving groups, such as the acetate, tosylate, mesylate or triflate group, are suitable as nucleofuge groups Nu in the compound R³—X-Nu that is used in the above-described process. The compounds R³—X-Nu thus belong, for example, to the classes of carboxylic acid, sulfonic acid, or sulfinic acid halides or the mixed anhydrides of these acids, as well as to the esters of chloroformic acid, toluenesulfonic acid, methylsulfonic acid and trifluoromethylsulfonic acid.

If, in the above-described process A, group X has the meaning of a bond, the nucleofuge group is a halogen atom (preferably a bromine or iodine atom). In this case, the reaction is run under copper catalysis.

Another process according to the invention (process B) for the production of compounds of general formula (I) consists in that a compound of general formula (II), as described above, is reacted with a compound of general formula R³—CHO, and the imine that is produced is reduced.

Another process according to the invention (process C) for the production of compounds of general formula (I) consists in that a compound of general formula (II), as described above, is reacted with phosgene or thiophosgene, and the isocyanate or isothiocyanate that is produced is then reacted with compounds of general formula R³—OH or R³—NH₂ to form a compound of general formula (I).

Compounds of general formula (II) can be obtained according to a process in which the 1-tetralone derivatives (V) that are known in the prior art are converted by selective oxidation into the 2-hydroxy-1-tetralone derivatives of general formula (IV), which then are converted into the corresponding oximes (III) and are catalytically reduced with hydrogen, by which the compounds of general formula (II) are obtained, in which substituent R⁴ is a hydroxy group. The reaction sequence is shown below in Diagram 1. The conditions for the individual process steps are familiar to one skilled in the art.

The tetralone derivatives of general formula (V) are accessible, for example, analogously to the processes described in WO 03/082827 and WO 2005/003098. 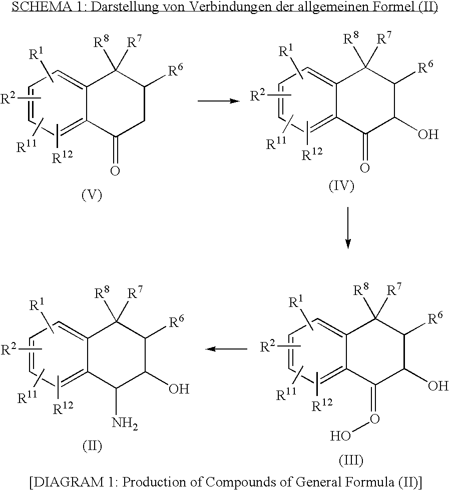
Biological Activity

The anti-inflammatory action of the compounds of general formula (I) is tested in the animal experiment by tests in the croton oil-induced inflammation in rats and mice (J. Exp. Med. (1995), 182, 99-108). To this end, croton oil in ethanolic solution is applied topically to the animals' ears. The test substances are also applied topically or systemically at the same time or two hours before the croton oil. After 16-24 hours, the ear weight is measured as a yardstick for inflammatory edema, the peroxidase activity as a yardstick for the invasions of granulocytes, and the elastase activity as a yardstick for the invasion of neutrophilic granulocytes. In this test, the compounds of general formula (I) inhibit the three above-mentioned inflammation parameters both after topical administration and after systemic administration.

The binding of the substances to the glucocorticoid receptor (GR) and other steroid-hormone receptors (mineral corticoid receptor (MR), progesterone receptor (PR) and androgen receptor (AR)) is examined with the aid of recombinantly produced receptors. Cytosol preparations of Sf9 cells, which had been infected with recombinant baculoviruses that code for the GR, are used for the binding studies. In comparison to the reference substance [³H]-dexamethasone, the substances show a high affinity to the GR.

The GR-mediated inhibition of the transcription of cytokines, adhesion molecules, enzymes and other pro-inflammatory factors is considered to be an essential molecular mechanism for the anti-inflammatory action of glucocorticoids. This inhibition is produced by an interaction of the GR with other transcription factors, e.g., AP-1 and NF-kappa-B (for a survey, see Cato, A. C. B. and Wade, E., BioEssays 18, 371-378, 1996).

The compounds of general formula (I) according to the invention inhibit the secretion of cytokine IL-8 into the human monocyte cell line THP-1 that is triggered by lipopolysaccharide (LPS). The concentration of the cytokines was determined in the supernatant by means of commercially available ELISA kits.

One of the most frequent undesirable actions of a glucocorticoid therapy is the so-called “steroid diabetes” [cf., Hatz, H. J., Glucocorticoide: Immunologische Grundlagen, Pharmakologie und Therapierichtlinien, [Glucocorticoids: Immunological Principles, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). A key enzyme of the catabolic metabolism in the liver is the tyrosinamino transferase (TAT). The activity of this enzyme can be determined from liver homogenates by photometry and represents a good measurement of the undesirable metabolic actions of glucocorticoids. To measure the TAT induction, the animals are sacrificed 8 hours after the test substances are administered, the livers are removed, and the TAT activity is measured in the homogenate. In this test, at doses in which they have an anti-inflammatory action, the compounds of general formula (I) induce little or no tyrosinamino transferase.

Medical Indications

Because of their anti-inflammatory action, and additional anti-allergic, immunosuppressive and antiproliferative action, the compounds of general formula (I) according to the invention can be used as medications for treatment or prophylaxis of the following pathologic conditions in patients, in particular mammals and preferably humans: In this case, the term. “DISEASE” stands for the following indications:
(i) Lung diseases that are accompanied by inflammatory, allergic and/or proliferative processes:
   Chronic, obstructive lung diseases of any origin, primarily bronchial asthma
   Bronchitis of different origins
   All forms of restrictive lung diseases, primarily allergic alveolitis
   All forms of pulmonary edema, primarily toxic pulmonary edema
   Sarcoidoses and granulomatoses, especially Boeck's disease
(ii) Rheumatic diseases/autoimmune diseases/joint diseases that are accompanied by inflammatory, allergic and/or proliferative processes:
   All forms of rheumatic diseases, especially rheumatoid arthritis, acute rheumatic fever, polymyalgia rheumatica
   Reactive arthritis
   Inflammatory soft-tissue diseases of other origins
   Arthritic symptoms in the case of degenerative joint diseases (arthroses)
   Traumatic arthritides
   Collagenoses of any origin, e.g., systemic lupus erythematodes, sclerodermia, polymyositis, dermatomyositis, Sjögren's syndrome, Still's syndrome, Felty's syndrome
(iii) Allergies that are accompanied by inflammatory and/or proliferative processes:
   All forms of allergic reactions, e.g., Quincke's edema, hay fever, insect bites, allergic reactions to pharmaceutical agents, blood derivatives, contrast media, etc., anaphylactic shock, urticaria, contact dermatitis
(iv) Vascular inflammations (vasculitides)
   Panarteritis nodosa, temporal arteritis, erythema nodosum
(v) Dermatological diseases that are accompanied by inflammatory, allergic and/or proliferative processes:
   Atopic dermatitis (primarily in children)
   Psoriasis
   Pityriasis rubra pilaris
   Erythematous diseases, triggered by different noxae, e.g., radiation, chemicals, burns, etc.
   Bullous dermatoses
   Diseases of the lichenoid group
   Pruritis (e.g., of allergic origin)
   Seborrheal eczema
   Rosacea
   Pemphigus vulgaris
   Erythema exudativum multiforme
   Balanitis
   Vulvitis
   Hair loss such as alopecia areata
   Cutaneous T-cell lymphoma
(vi) Kidney diseases that are accompanied by inflammatory, allergic and/or proliferative processes:
   Nephrotic syndrome
   All nephritides
(vii) Liver diseases that are accompanied by inflammatory, allergic and/or proliferative processes:
   Acute liver cell decomposition
   Acute hepatitis of different origins, e.g., viral, toxic, pharmaceutical agent-induced
   Chronic aggressive hepatitis and/or chronic intermittent hepatitis
(viii) Gastrointestinal diseases that are accompanied by inflammatory, allergic and/or proliferative processes:
   Regional enteritis (Crohn's disease)
   Colitis ulcerosa
   Gastritis
   Reflux esophagitis
   Ulcerative colitis of other origins, e.g., native sprue
(ix) Proctologic diseases that are accompanied by inflammatory, allergic and/or proliferative processes:
   Anal eczema
   Fissures
   Hemorrhoids
   Idiopathic proctitis
(x) Eye diseases that are accompanied by inflammatory, allergic and/or proliferative processes:
   Allergic keratitis, uveitis, iritis
   Conjunctivitis
   Blepharitis
   Optic neuritis
   Chorioiditis
   Sympathetic ophthalmia
(xi) Diseases of the ear-nose-throat area that are accompanied by inflammatory, allergic and/or proliferative processes:
   Allergic rhinitis, hay fever
   Otitis extema, e.g., caused by contact dermatitis, infection, etc.
   Otitis media
(xii) Neurological diseases that are accompanied by inflammatory, allergic and/or proliferative processes:
   Cerebral edema, primarily tumor-induced cerebral edema
   Multiple sclerosis
   Acute encephalomyelitis
   Meningitis
   Various forms of convulsions, e.g., infantile nodding spasms
(xiii) Blood diseases that are accompanied by inflammatory, allergic and/or proliferative processes:
   Acquired hemolytic anemia
   Idiopathic thrombocytopenia
(xiv) Tumor diseases that are accompanied by inflammatory, allergic and/or proliferative processes:
   Acute lymphatic leukemia
   Malignant lymphoma
   Lymphogranulomatoses
   Lymphosarcoma
   Extensive metastases, mainly in breast, bronchial and prostate cancers
(xv) Endocrine diseases that are accompanied by inflammatory, allergic and/or proliferative processes:
   Endocrine orbitopathy
   Thyreotoxic crisis
   De Quervain's thyroiditis
   Hashimoto's thyroiditis
   Basedow's disease
(xvi) Organ and tissue transplants, graft-versus-host disease
(xvii) Severe shock conditions, e.g., anaphylactic shock, systemic inflammatory response syndrome (SIRS)
(xviii) Vomiting that is accompanied by inflammatory, allergic and/or proliferative processes:
   e.g., in combination with a 5-HT3 antagonist in cytostatic-agent-induced vomiting
(xix) Pain of inflammatory origin, e.g., lumbago
(xx) Substitution therapy in:
   Innate primary suprarenal insufficiency, e.g., congenital adrenogenital syndrome
   Acquired primary suprarenal insufficiency, e.g., Addison's disease, autoimmune adrenalitis, meta-infective tumors, metastases, etc.
   Innate secondary suprarenal insufficiency, e.g., congenital hypopituitarism
   Acquired secondary suprarenal insufficiency, e.g., meta-infective tumors, etc.

Pharmaceutical agents that contain stereoisomers of general formula I show special effectivness in the case of the following diseases:
1. Lung diseases
2. Rheumatic diseases/autoimmune diseases
3. Dermatological diseases
4. Degenerative joint diseases
5. Vascular inflammations
6. Graft-versus-host disease
7. Severe shock conditions
8. Vomiting that is accompanied by inflammatory, allergic and/or proliferative processes
9. Inflammation-induced pain.

Moreover, the compounds of general formula (I) according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

All previously mentioned indications are described in detail in Hatz, H. J., Glucocorticoide: Immunologies Grundlagen, Pharmakologie und Therapierichtlinien, Wissenschaftliche Verlagsgesellschaft mbH, Stuttgart, 1998.

For the therapeutic action in the above-mentioned pathologic conditions, the suitable dose varies and depends on, for example, the active strength of the compound of general formula (I), the patient (e.g., size, weight, sex, etc.), the type of administration, and the type and severity of the conditions that are to be treated, as well as the use as a prophylactic agent or therapeutic agent.

The invention relates to the use of the claimed compounds for the production of a pharmaceutical composition.

In addition, the invention provides:
(i) The use of one of the compounds of general formula (I) according to the invention or mixture thereof for the production of a pharmaceutical composition for treating or preventing inflammatory processes, and in particular for treating a DISEASE (as defined above);
(ii) A process for treating or preventing inflammatory processes, in particular for treating a DISEASE (as defined above), said process comprising an administration of a pharmaceutically effective amount of a compound of general formula (I), whereby this amount relieves or suppresses the disease or the symptoms, and whereby the compound is given to a patient, preferably a mammal, in particular a human, who requires such a treatment;
(iii) A pharmaceutical composition for anti-inflammatory action, in particular for treating a DISEASE (as defined above), whereby the composition comprises one of the compounds according to the invention or mixture thereof and optionally at least one pharmaceutical adjuvant and/or vehicle.

In general, satisfactory results can be expected in animals when the daily doses comprise a range of 1 μg to 100,000 μg of the compound according to the invention per kg of body weight. In the case of larger mammals, for example the human, a recommended daily dose lies in the range of 1 μg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. For treating acute shock (e.g., anaphylactic shock), single doses can be given that are considerably above the above-mentioned doses.

The formulation of the pharmaceutical preparations based on the new compounds is carried out in a way that is known in the art by the active ingredient being processed with the vehicles, fillers, substances that influence decomposition, binding agents, moisturizers, lubricants, absorbents, diluents, flavoring correctives, coloring agents, etc., that are commonly used in galenicals and converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments, tinctures and gels in corresponding pharmaceutical preparations.

For topical application, formulations in gels, ointments, fatty ointments, creams, pastes, powders, suspensions, emulsions, and solutions are possible. The dosage of the compounds of general formula (I) should be 0.01%-20% in these preparations to achieve a sufficient pharmacological action.

The invention also comprises the compounds of general formula (I) according to the invention as therapeutic active ingredients. In addition, the compounds of general formula (I) according to the invention are part of the invention as therapeutic active ingredients together with one or more pharmaceutically compatible and acceptable adjuvants and/or vehicles.

The compounds of general formula (I) according to the invention can optionally also be formulated and/or administered in combination with other active ingredients.

The invention therefore also relates to combination therapies or combined compositions, in which a compound of general formula (I) or a pharmaceutically acceptable salt thereof, or a pharmaceutical composition that contains a compound of general formula (I) or a pharmaceutically acceptable salt thereof, is administered either simultaneously (optionally in the same composition) or in succession together with one or more pharmaceutical agents for treating one of the above-mentioned pathologic conditions. For example, for treatment of rheumatoid arthritis, osteoarthritis, COPD (chronic obstructive lung disease), asthma or allergic rhinitis, a compound of general formula (I) of this invention can be combined with one or more pharmaceutical agents for treating such a condition. When such a combination is administered by inhalation, the pharmaceutical agent that is to be combined can be selected from the following list:
A PDE4 inhibitor including an inhibitor of the PDE4D isoform,
A selective β.sub2.adrenoceptor agonist, such as, for example, metaproterenol, isoproterenol, isoprenaline, albuterol, salbutamol, formoterol, salmeterol, terbutaline, orcipresnaline, bitolterol mesylate, pirbuterol or indacaterol;
A muscarine receptor antagonist (for example, an M1, M2 or M3 antagonist, such as, for example, a more selective M3 antagonist), such as, for example, ipratropium bromide, tiotropium bromide, oxitropium bromide, pirenzepine or telenzepine;
A modulator of the chemokine receptor function (such as, for example, a CCR1 receptor antagonist); or
An inhibitor of the p38 kinase function.

For another subject of this invention, such a combination with a compound of general formula (I) or a pharmaceutically acceptable salt thereof is used for treatment of COPD, asthma or allergic rhinitis and can be administered by inhalation or orally in combination with xanthine (such as, for example, aminophylline or thyeophylline), which also can be administered by inhalation or orally.

### EXAMPLES

### Production of 4,4-Dimethyl-1-pyrimidin-5-ylamino-1,2,3,4-tetrahydronaphthalen-2-ol

5,5-Dimethyldihydrofuran-2-one (J. Am. Chem. Soc. 1947, 69, 2322-2324), 4,4-dimethyl-3,4-dihydro-2H-naphthalen-1-one (J. Am. Chem. Soc. 1947, 69, 2322-2324) and 2-hydroxy-4,4-dimethyl-3,4-dihydro-2H-naphthalen-1-one (J. Org. Chem. 1994, 59, 1184-1190) are produced according to the literature.

### A. 2-Hydroxy-4,4-dimethyl-3,4-dihydro-2H-naphthalen-1-one

0.87 g (5.0 mmol) of 4,4-dimethyl-3,4-dihydro-2H-naphthalen-1-one was dissolved in 20 ml of THF under argon, cooled to −74° C., and then 15.2 ml (0.5 M in toluene, 7.6 mmol) of potassium-bis-(trimethylsilyl)amide solution is added in drops thereto at −74 to −71.5° C., and stirring is continued for 30 minutes at −74° C. 2.3 g (7.6 mmol) of [(8,8-dichlorocamphoryl)sulfonyl]oxaziridine was dissolved in 30 ml of THF and added in drops to the reaction solution at −74 to −68° C.; in this case, the reaction mixture was colored dark red. The reaction was stirred for 75 minutes at −74° C. For working-up, the reaction mixture was carefully mixed with saturated sodium bicarbonate solution and then extracted three times with ethyl acetate. The combined organic phases were washed with saturated sodium chloride solution, dried on sodium sulfate, filtered off and concentrated by evaporation in a vacuum. 3.07 g of viscous yellow oil was obtained as a crude product, which was purified by column chromatography with ethyl acetate/hexane: 539 mg (57%) of light yellow, viscous oil.

¹H-NMR (300 MHz, CDCl₃): δ/ppm=1.46 (s, 6H), 2.05 (dd, 1H), 2.30 (dd, 1H), 3.74 (s, 1H), 4.60 (dd, 1H), 7.34 (t, 1H), 7.44 (d, 1H), 7.58 (t, 1H), 8.00 (d, 1H).

### B. 2-Hydroxy-4,4-dimethyl-3,4-dihydro-2H-naphthalen-1-one oxime

525 mg (2.76 mmol) of 2-hydroxy-4,4-dimethyl-3,4-dihydro-2H-naphthalen-1-one was dissolved in 10 ml of pyridine, cooled to −14° C., and 348 mg (5.53 mmol) of hydroxylamine-hydrochloride was added, heated slowly to room temperature, and stirred for 48 hours. For working-up, pyridine was distilled off in a rotary evaporator, the residue was mixed with 20 ml of saturated sodium chloride solution and 20 ml of citric acid solution (10%) and extracted three times with 30 ml each of ethyl acetate. The combined organic phases were washed with saturated sodium chloride solution, dried on sodium sulfate, filtered off and concentrated by evaporation. 608 mg of crude product, which was purified by column chromatography with hexane/ethyl acetate, was obtained: 402 mg (71%) of colorless, viscous oil.

¹H-NMR (300 MHz, CDCl₃): δ/ppm=1.23 (s, 3H), 1.44 (s, 3H), 1.86 (dd, 1H), 2.09 (dd, 1H), 5.21 (dd, 1H), 7.21-7.27 (m, 1H), 7.37-7.39 (m, 2H), 7.82 (d, 1H).

### C. 1-Amino-4,4-dimethyl-1,2,3,4-tetrahydronaphthalen-2-ol

381 mg (1.86 mmol) of 2-hydroxy-4,4-dimethyl-3,4-dihydro-2H-naphthalen-1-one oxime was dissolved in 45 ml of methanol, mixed with 78 mg of palladium on activated carbon (10%) and mixed with 0.6 ml of concentrated hydrochloric acid. The flask was evacuated several times, flushed with hydrogen, and then stirred for 6 hours at room temperature under hydrogen. For working-up, the catalyst was filtered off on Celite, rewashed with methanol, the filtrate was mixed with 5 ml of saturated sodium bicarbonate solution and concentrated by evaporation in a rotary evaporator. The residue was mixed with saturated sodium bicarbonate solution and ethyl acetate, and the organic phase was separated. The aqueous phase was re-extracted twice with ethyl acetate. The combined organic phases were washed with sodium chloride solution, dried on sodium sulfate, filtered off and concentrated by evaporation in a vacuum: 320 mg (90%) of product.

¹H-NMR (300 MHz, CDCl₃): δ/ppm=1.30-1.35 (m, 3H), 1.40 (s, 3H), 1.76-2.05 (m, 2H), 2.24 (bs, 3H), 3.63-4.09 (m, 2H), 7.18-7.53 (m, 3H), 7.49-7.53 (m, 1H).

### D. 4,4-Dimethyl-1-pyrimidin-5-ylamino-1,2,3,4-tetrahydronaphthalen-2-ol

57 mg (0.30 mmol) of 1-amino-4,4-dimethyl-1,2,3,4-tetrahydronaphthalen-2-ol, 32 mg (0.20 mmol) of 5-bromopyridine, 3.8 mg (0.02 mmol) of copper iodide, 7.73 mg (0.04 mmol) of N,N-diethyl-2-hydroxy-benzamide and 84.9 mg (0.4 mmol) of potassium phosphate were weighed in a microwave vessel, flushed with argon, then 150 μl of DMF was added, the vessel was sealed and stirred for 21 hours at 90° C. For working-up, the reaction mixture was cooled to room temperature, mixed with 2 ml of ethyl acetate, and 0.5 ml of (33%) ammonium hydroxide solution and 5 ml of water were added. It was shaken out twice with ethyl acetate, the organic phases were combined, washed with sodium chloride solution, dried on sodium sulfate and concentrated by evaporation in a vacuum. 97 mg of crude product was purified by column chromatography (column material: amine phase, ethyl acetate): 31 mg (58%). By preparative HPLC, the desired product was obtained.

¹H-NMR (300 MHz, DMSO-d₆): δ/ppm=1.29 (s, 3H), 1.34 (s, 3H), 1.77-1.87 (m, 2H), 3.87 (bs, 1H), 4.42 (t, 1H), 5.00 (d, 1H), 6.47 (d, 1H), 7.14 (t, 1H), 7.22-7.24 (m, 2H), 7.38 (d, 1H), 8.18 (s, 2H), 8.33 (s, 1H).

According to the above-indicated instructions, the following compounds are also obtained:

4,4-Dimethyl-7-methoxy-1-pyrimidin-5-ylamino-1,2,3,4-tetrahydronaphthalen-2-ol,

8-Fluoro-4,4-Dimethyl-5-methoxy-1-pyrimidin-5-ylamino-1,2,3,4-tetrahydronaphthalen-2-ol,

4,4-Dimethyl-1-naphth-1-ylamino-1,2,3,4-tetrahydronaphthalen-2-ol, and

4,4-Dimethyl-1-quinolin-5-ylamino-1,2,3,4-tetrahydronaphthalen-2-ol.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No.10 2005 017 286.5, filed Apr. 14, 2005, and U.S. Provisional Application Ser. No. 60/671,108, filed Apr. 14, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula (I), 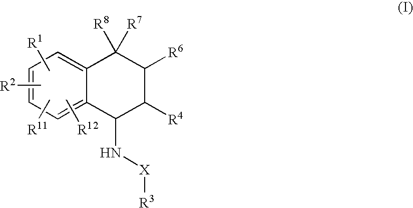
in which
R¹ and R², independently of one another, are a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, a cyano group, a nitro group, or an —NR⁹R^{9a} group,
or R¹ and R² together form a group that is selected from the groups —O—(CH₂)ₙ-O—, —O—(CH₂)ₙ-CH₂—, —O—CH═CH—, —(CH₂)ₙ₊₂-, —NH—(CH₂)ₙ₊₁-, —N(C₁-C₃-alkyl)-(CH₂)ₙ₊₁-, and —NH—N═CH—, whereby n=1 or 2, and the terminal oxygen atoms and/or carbon atoms and/or nitrogen atoms are linked to directly adjacent ring-carbon atoms,
R¹¹ is a hydrogen atom, a hydroxy group, a halogen atom, a cyano group, an optionally substituted (C₁-C₁₀)-alkyl group, a (C₁-C₁₀)-alkoxy group, a (C₁-C₁₀)-alkylthio group, or a (C₁-C₅)-perfluoroalkyl group,
R¹² is a hydrogen atom, a hydroxy group, a halogen atom, a cyano group, an optionally substituted (C₁-C₁₀)-alkyl group, or a (C₁-C₁₀)-alkoxy group,
R³ is a (C₁-C₁₀)-alkyl group that optionally is substituted by 1 to 3 hydroxy groups, 1 to 3 halogen atoms, and/or 1 to 3 (C₁-C₅)-alkoxy groups, an optionally substituted (C₃-C₇)-cycloalkyl group, an optionally substituted heterocyclyl group, an optionally substituted aryl group, a monocyclic or bicyclic heteroaryl group that optionally is substituted by one or more groups, which independently of one another, are selected from
(C₁-C₅)-alkyl groups, which themselves optionally can be substituted by 1 to 3 hydroxy groups or 1 to 3 —COOR¹³ groups,
(C₁-C₅)-alkoxy groups,
halogen atoms, hydroxy groups, —NR⁹R^{9a} groups, and
exomethylene groups,
and that contains 1 to 4 nitrogen atoms and/or 1 to 2 oxygen atoms and/or 1 to 2 sulfur atoms and/or 1 to 2 keto groups, whereby this group is linked via any position to group X and optionally can be hydrogenated at one or more sites,
R⁴ is a hydroxy group, an —OR¹⁰ group or an —O(CO)R¹⁰ group,
R⁶ is a hydrogen atom, a halogen atom, or an optionally substituted (C₁-C₁₀)-alkyl group,
R⁷ and R⁸, independently of one another, mean a hydrogen atom, a halogen atom, an optionally substituted (C₁-C₁₀)-alkyl group, a cyano group, together a (C₁-C₁₀)-alkylidene group or together with the carbon atom of the tetrahydronaphthalene system an optionally substituted (C₃-C₆)-cycloalkyl ring; or
R⁶ and R⁷ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms; or
R¹ and R⁸ together form an annelated five- to eight-membered, saturated or unsaturated carbocyclic compound or heterocyclic compound, which optionally is substituted by 1 to 2 keto groups, 1 to 2 (C₁-C₅)-alkyl groups, 1 to 2 (C₁-C₅)-alkoxy groups, and/or 1 to 4 halogen atoms;
R⁹ and R^{9a}, independently of one another, are a hydrogen atom, a (C₁-C₅)-alkyl group or —(CO)—(C₁-C₅)-alkyl,
R¹⁰ means a (C₁-C₁₀)-alkyl group or any hydroxy protective group,
R¹³ means a hydrogen atom or a (C₁-C₅)-alkyl group, and
X means a bond or a group —C(═O)—, —C(═S)—, —C(═O)—NH—, —C(═S)—NH—, —S(O)ₘ-(whereby m=1 or 2), —C(═O)—O—, —C(═S)—O— or a group —(CH₂)ₚ- (whereby p=1, 2 or 3), whereby, if X contains a carbonyl- or thiocarbonyl function, this function is bonded to the group —NH— in general formula (I),
in the form of any stereoisomer or a mixture of stereoisomers; or as a pharmacologically harmless salt or derivative.

2. Compounds according to claim 1, whereby
X is a bond or a group —C(═O)—, —C(═O)—NH—, —SO₂— or —CH₂—.

3. Compounds according to claim 1, whereby
R⁴ is a hydroxy group or a group —OR¹⁰.

4. Compounds according to one of the preceding claim 1, whereby
R⁶ is a hydrogen atom.

5. Compounds according to claim 1, whereby
R⁷ and R⁸ in each case represent a methyl group, or together with the carbon atom of the tetrahydronaphthalene system form a cyclopropyl group.

6. Compounds according to claim 1, whereby
R³ means an optionally substituted aryl or heteroaryl group.

7. Compounds according to claim 6, whereby the aryl or heteroaryl group is selected from the group that consists of optionally substituted naphthyl, benzofuranyl, pyrazolo[1,5-a]pyridinyl, phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, chromanyl, isochromanyl, phthalazinyl, 1,7- or 1,8-naphthyridinyl, dihydroindolonyl, dihydroisoindolonyl, benzimidazole or indolyl groups.

8. Compounds according to claim 1 for the production of a pharmaceutical agent.

9. Use of a compound according to claim 1 for the production of a pharmaceutical composition for treating or preventing inflammatory processes.

10. Process for treating or preventing inflammatory processes in a patient, characterized in that a pharmaceutically effective amount of a compound of general formula (I) claim 1 is administered to a patient who requires such a treatment or prevention.

11. Pharmaceutical preparations that contain at least one compound claim 1, as well as one or more pharmaceutically compatible vehicles and/or adjuvants.

12. Process for the production of a compound of general formula (I) according to claim 1, wherein a compound of general formula (II) 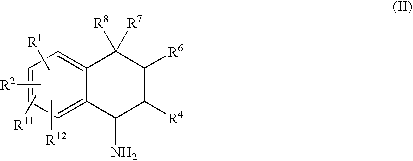
a) is reacted to form a compound of general formula (I) by reaction with a compound that is selected from compounds of general formulas R³—X-Nu (whereby Nu represents a nucleofuge group), R³—N═C═O or R³—N═C═S, and optionally then exchange of a carbonyl-oxygen atom for sulfur, or
b) is reacted with a compound of general formula R³—CHO, and the imine that is produced is reduced, or
c) is reacted with phosgene or thiophosgene, and the isocyanate or isothiocyanate that is produced is then reacted to form a compound of general formula (I) with compounds of general formula R³—OH or R³—NH₂;
whereby the substituents R¹ to R¹² and X have the meanings that are indicated in claim 1.

13. Compounds of general formula (II) 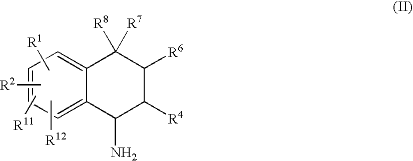
whereby substituents R¹ to R¹² have the meanings that are indicated in claim 1.

14. A method of pre-paring a compound of general formula (I) comprising reacting a compound of claim 13.

15. Compounds of general formula (IV), 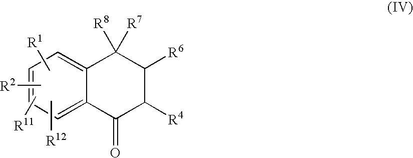
whereby substituents R¹ to R¹² have the meanings that are indicated in claim 1.

16. A method of preparing a compound of general formula (I) comprising reacting a compound of claim 15.
